(19) United States
(12) Patent Application Publication
(10) Pub. No.: US 2021/0267854 A1
(43) Pub. Date: Sep. 2, 2021

(54) SKIN HYDRATION COMPOSITION

(71) Applicant: MASTER DYNAMIC LIMITED, Hong Kong (CN)

(72) Inventors: Ka Wing CHENG, Hong Kong (CN); Wng Chi TANG, Hong Kong (CN); Koon Chung HUI, Hong Kong (CN)

(73) Assignee: MASTER DYNAMIC LIMITED, Hong Kong (CN)

(21) Appl. No.: 17/253,339

(22) PCT Filed: Jun. 21, 2019
(86) PCT No.: PCT/CN2019/092171

§ 371 Date: Dec. 17, 2020

(30) Foreign Application Priority Data
Jun. 22, 2018 (HK) 18108034.1

(51) Int. Cl. A61K 8/02; A61K 8/19; A61Q 19/00; A61K 8/86; A61K 45/06

(52) U.S. Cl. CPC A61K 8/0245; A61K 8/19; B82Y 5/00; A61K 8/86; A61K 45/06; A61Q 19/00

(57) ABSTRACT

A topically administrable moisturising composition for moisturising the skin of a subject, said moisturising composition comprising (i) a plurality of conjugates wherein each conjugate is comprised of a nanodiamond and a plurality of water molecules, and (ii) a carrier media for topical administration.

### TECHNICAL FIELD

The present invention relates to a topically administrable moisturising composition for moisturising the skin, in particular the present invention provides a formulation for hydrating the skin subjects.

### BACKGROUND OF THE INVENTION

Skin hydration is crucial to the health of skin or dermis of a subject. Without sufficient hydration of skin, several dermal diseases can occur to a subject.

Some over-the-counter (OTC) commercially available topical medications for eczema are essentially moisturiser products that mainly moisturise the skin of a subject and prevent water evaporation from the dermis.

However, frequent application of such moisturiser products to the skin of a subject is required.

Such moisturiser products applied on skin surface of a subject are easily worn away or wiped off, for example by the friction with clothing, contact with environment, washing of hands or the like.

The need of frequent application of a moisturizer product causes inconvenience to the subject. As will be understood, a subject cannot simply overapply a moisturizer product to seek to provide a longer moisturising effect, as inevitably the product still may be wiped off, as well will adhere to clothing or other articles, and also the effect will be saturation as well as unwanted side effect which may occur, and such over-application is practically inconvenient in any event.

Furthermore, some topically applied medicaments require the dermis of a subject to remain hydrated for effective bioactivity of the active pharmaceutical ingredient (API) on the dermis of a subject, and ease of removable of the medicament, such as a cream, ointment, lotion or the like, will result in reduced efficacy of the medicament.

Therefore, to improve the quality of a subject, an improved moisturizing product is required which reduces the effect of the product being wiped and being worn away and increased moisturizing effect is required.

### OBJECT OF THE INVENTION

It is an object of the present invention to provide a moisturising composition which overcomes or at least partly ameliorates at least some deficiencies as associated with the prior art.

### SUMMARY OF THE INVENTION

In a first aspect, the present invention provides a topically administrable moisturising composition for moisturising the skin of a subject, said moisturising composition comprising (i) a plurality of conjugates wherein each conjugate is comprised of a nanodiamond and a plurality of water molecules, and (ii) a carrier media for topical administration.

The water molecules are adsorbed to the nanodiamond, wherein the water molecules may be adsorbed to the nanodiamond by way of a functional group on the surface of the nanodiamond. The functional group may be a carboxylate group (—COOH) or a hydroxyl group (—OH).

The nanodiamonds may further include a functional polymer thereon, for further moisturising the skin of a subject. The functional may be polyethylene glycol (PEG).

The nanodiamond is preferably sized so as to prevent permeation through the stratum corneum of the skin of the subject. The nanodiamond is preferably sized greater than 50 nm so as to prevent permeation through the stratum corneum of the skin of the subject

The carrier may be selected from the group including cream, an ointment, a gel, a foam, a lotion, a spray or the like.

The composition may further include at least one active pharmaceutical ingredient for providing a pharmaceutical effect to the subject. The at least one pharmaceutical ingredient provides a pharmaceutical effect to the skin of the subject.

In a second aspect, the present invention provides a method of moisturising the skin of a subject, wherein said method includes the step of applying the topically administrable moisturising composition of the first aspect to the skin of a subject.

In a third aspect, the present invention provides the use of the topically administrable moisturising composition of the first aspect for the manufacture of a medicament for the moisturising of the skin of a subject.

In a fourth aspect, the present invention provides a topically administrable moisturising composition according to the first aspect, for use in the moisturising of the skin of a subject.

### DETAILED DESCRIPTION OF THE INVENTION

The present inventors have identified shortcomings of the prior art, and upon identification of the problems with the prior art, have provided a product which overcomes the problems of the prior art, and provides a product which is more consistent and reliable.

The present invention provides an improved moisturizing product and an improvement to medicaments requiring moisturization of the dermis during application, and also which is of assistance in treating and preventing skin disorders and afflictions to subjects.

In embodiments of the invention, the moisturizing product may include one or more active pharmaceutical ingredients for topical administration to the skin of a subject.

As a moisturizing product, the present invention provides a composition for assisting in the prevention or treatment of eczema. Atopic dermatitis, which is also known as atopic eczema or eczema, is a very common dermal disease for which a subject may suffer from red and itchy skin.

Other symptoms of eczema may vary between individuals, which includes dry skin, itching, red patches, raised bumps on the skin, thickened or scaly skin, which often results in bleeding of skin due to scratching by the subject. Whilst subjects are often aware that scratching of skin exacerbates the symptoms of eczema, it is very difficult for a subject to refrain from scratching, and such scratching may be considered involuntary and also can occur when the subject is sleeping.

In addition to the inconvenience, and at times debilitating effects of such diseases, subjects afflicted with such diseases often suffer psychological effects by being uncomfortable or self-conscious in the presence of other persons, as well as be exposes to social rejection or at times ridicule.

As such, it is important that such diseases are effectively and efficiently treated, so as to at least render an affliction manageable or reduced to a manageable level, or where possible treated an eradicated.

The exact cause of eczema remains unknown, however it is thought to be related to genetic factors, as well as the environment a subject is exposed to as well as some degree of compromise or malfunction of the subject's immune system.

It is known that triggers factors of eczema are found to be irritants such as stress, nickel metal, cigarette smoke, chemicals used in daily cleaning products used cleaners, insects, soap products and even some skin care products.

Although the underlying cause of eczema may be unclear, there are ways in which to prevent the triggers of eczema.

Beside the above identified and known irritants which can cause eczema, skin dryness is reported to be one of the most predominant irritants which can trigger eczema.

As such, appropriate moisturising the skin of a subject is of primary importance.

In accordance with the present invention, in order to prevent the skin applied topical medications being worn away and increased moisturizing effect, a topically administrable moisturising composition for moisturising the skin of a subject is provided, comprising:

(i) a plurality of conjugates wherein each conjugate is comprised of a nanodiamond and a plurality of water molecules, and

(ii) a carrier media for topical administration.

Nanodiamonds (NDs) have been intensively studied for cytotoxicity. As diamond is carbon in nature with very low reactivity, and is very biocompatible.

On the surface of nanodiamonds, there are various functional groups for terminations, such as hydrogen (—H), carboxylate group (—COOH), hydroxyl group (—OH), etc.

These functional groups provide the surface of nanodiamonds ability to bond with biomolecules or chemicals. With suitable combination of surface functional groups of nanodiamond, biomolecules or chemicals can also adsorb on the surface physically without bonding.

For carboxylate and hydroxyl groups, these groups are hydrophilic. As such, and in accordance with the invention, water molecules can be attached on the nanodiamond surface by way of these functional groups.

Such nanodiamonds having such functional groups allow for dissolution in water. As nanoparticles have a large surface area, hydrophilic nanodiamonds can carry much moisture by way of water molecules on the surface of the nanodiamonds.

Such nanodiamonds can also be applied with a functional polymer, such as polyethylene glycol (PEG), on the surface to further enhance the moisturizing effect of the composition.

Suitably sized of nanodiamonds can be engineered for use in accordance with the invention. Different sized nanodiamonds have been demonstrated to have different levels of cell and tissue permeabilities.

Studies have shown that carboxylated nanodiamonds with the size of approximately 50 nm could not permeate through the stratum corneum, the outermost layer of the epidermis.

Further, the nanodiamonds can remain in the hair follicles of a subject, which also enables control of moisture release physically by nanodiamonds of the present invention.

Accordingly, when the moisturiser composition of the present invention with suitably sized nanodiamonds carrying thereon water molecules thereon is applied on the skin of a subject, the nanodiamonds are prevented from penetrating the skin of a subject and are prevented from entering bloodstream.

Rather, the nanodiamonds will remain on skin of the subject, possibly in the hair follicles, and release moisture throughout the time to the skin of the subject and the therapeutic effect thereof. As nanodiamonds can fit into hair follicles, moisture can be delivered topically to a subject without concern of being washed away easily or wiped from the skin of a subject.

In accordance with the invention, the carrier for forming the composition may be a cream, an ointment, a gel, a foam, a lotion, a spray or the like.

A cream may be considered an emulsion of oil and water in approximately equal proportions, and which penetrates stratum corneum outer layer of skin well. An ointment is a combination of oil and water, typically 80% oil and 20% water, can provide barrier against moisture loss. A gel is a material that liquefies upon contact with the skin. A paste is a combination of three agents, oil, water, and powder; an ointment in which a powder is suspended.

In embodiments of the invention, the composition can further include at least one active pharmaceutical ingredient for providing a pharmaceutical effect to the subject, which provides a pharmaceutical effect to the skin of the subject.

The topically administrable moisturising composition of the present invention can be used to moisturise the skin of a subject for general skin health maintenance, as well as for preventing or treating dermatological disorders such as eczema in the illustrative example above.

The composition can include various excipients as which may be used in creams, ointments, gels, foams, lotions, pastes and spray which, although not specifically recited, will be understood by those skilled in the art to be inherently applicable to the present invention.

The present invention provides significant advantages over the prior art, by maintaining moisture at an appropriate position in the skin for absorption and hydration of the skin of the subject.

Suitably sized nanodiamonds allow for the holding of water molecules at the surface of the skin and within hair follicles, thus assisting in providing a topically administrable moisturising composition which need not be reapplied as frequently as those of the prior art, whilst having resistance to removable from the skin by wiping, washing and rubbing by clothing.

1. A topically administrable moisturising composition for moisturising the skin of a subject, said moisturising composition comprising:
(i) a plurality of conjugates wherein each conjugate is comprised of a nanodiamond and a plurality of water molecules, and
(ii) a carrier media for topical administration.

2. The topically administrable moisturising composition according to claim 1, wherein the water molecules are adsorbed to the nanodiamond.

3. The topically administrable moisturising composition according to claim 2, wherein the water molecules are adsorbed to the nanodiamond by way of a functional group on the surface of the nanodiamond.

4. The topically administrable moisturising composition according to claim 3, wherein the functional group is a carboxylate group (—COOH) or a hydroxyl group (—OH).

5. The topically administrable moisturising composition according to claim 1, wherein the nanodiamonds further include a functional polymer thereon, for further moisturising the skin of a subject.

6. The topically administrable moisturising composition according to claim 1, wherein the nanodiamonds further include polyethylene glycol (PEG) thereon, for further moisturising the skin of a subject.

7. The topically administrable moisturising composition according to claim 1, wherein the nanodiamond is sized so as to prevent permeation through the stratum corneum of the skin of the subject

8. The topically administrable moisturising composition according to claim 1, wherein the nanodiamond is sized greater than 50 nm so as to prevent permeation through the stratum corneum of the skin of the subject

9. The topically administrable moisturising composition according to claim 1, wherein the carrier is selected from the group including cream, an ointment, a gel, a foam, a paste, a lotion, a spray or the like.

10. The topically administrable moisturising composition according to claim 1, wherein the composition further includes at least one active pharmaceutical ingredient for providing a pharmaceutical effect to the subject.

11. The topically administrable moisturising composition according to claim 10, wherein the at least one pharmaceutical ingredient provides a pharmaceutical effect to the skin of the subject.

12. A method of moisturising the skin of a subject, wherein said method includes the step of applying the topically administrable moisturising composition of claim 1 to the skin of a subject.

13. The use of the topically administrable moisturising composition of claim 1 for the manufacture of a medicament for the moisturising of the skin of a subject.

14. The topically administrable moisturising composition according to claim 1, for use in the moisturising of the skin of a subject.
